(19) United States
(12) Patent Application Publication
(10) Pub. No.: US 2026/0320978 A1
(43) Pub. Date: Oct. 15, 2026

(54) BRAIN METASTASES DETECTION

(71) Applicant: Hoffmann-La Roche Inc., Little Falls, NJ (US)

(72) Inventors: Benjamin GUTIÉRREZ BECKER, Basel (CH); Damian Marek KUCHARSKI, Gliwice (PL); Bartosz Jakub MACHURA, Gliwice (PL); Jakub Robert NALEPA, Gliwice (PL); Jean Joseph Louis TESSIER, Muespach (FR)

(73) Assignee: Hoffmann-La Roche Inc., Little Falls, NJ (US)

(21) Appl. No.: 19/576,214

(22) Filed: Mar. 24, 2026

Related U.S. Application Data
(63) Continuation of application No. PCT/EP2024/078833, filed on Oct. 14, 2024.

(30) Foreign Application Priority Data
Oct. 16, 2023 (EP) 23203714.3

(51) Int. Cl. G06V 10/774; A61B 5/00; A61B 5/055; G06T 7/00

(52) U.S. Cl. CPC G06V 10/774; A61B 5/055; A61B 5/4064; G06T 7/0012; G06T 2207/10088; G06T 2207/20081; G06T 2207/30016; G06T 2207/30096; G06V 2201/031

(57) ABSTRACT

The present invention relates to systems and methods in the field of digital pathology. It is particularly, but not exclusively, concerned with systems and methods for detecting brain lesions, and in particular brain metastases, in brain digital images.

### FIELD OF INVENTION

The present invention relates to systems and methods in the field of digital pathology. It is particularly, but not exclusively, concerned with systems and methods for detecting brain lesions, and in particular brain metastases, in brain digital images.

### BACKGROUND TO THE INVENTION

Metastatic brain cancer is caused by cancer cells spreading to the brain from a different part of the body. The most common types of cancer that metastasize are lung cancers, breast cancers, melanomas, colon cancers, kidney cancers and thyroid cancers. Metastatic brain cancers are five times more common than primary brain tumors. Metastatic brain tumors can grow rapidly and can spread in different areas of the brain in parallel.

MRI scans of subjects with metastatic brain tumors show complex and heterogeneous features: metastases can be small or huge blobs, can appear like bright spots or dark areas, can be as few as a handful or as many as hundreds of lesions. In addition, subjects can present surgical cavities, necrosis, edema regions.

Several trained Deep-Learning (DL) models exist^{1,2,3,4,5,6,7,8,9,10} that are used to detect brain metastases in brain MRI scans. However, to develop said trained models, the available brain images datasets are randomly split into a train set and a test set without any constraint. Therefore said models do not perform well enough in detecting lesions characterized by heterogeneous features.

Thus there is a need for improved systems and methods for detecting brain lesions, and in particular brain metastases, in brain digital images.

### STATEMENTS OF INVENTION

The present invention relates to systems and methods in the field of digital pathology. It is particularly, but not exclusively, concerned with systems and methods for detecting brain lesions, and in particular brain metastases, in brain digital images. The methods can find particular use in the detection of brain metastases in digital images from MRI scans via machine-learning models. The methods can be used, among other applications, to detect brain lesions, to monitor and/or predict the evolution in time of brain lesions, to monitor and/or predict disease progression, to assess and/or predict the response of a patient to a treatment, to identify cohorts of patients with similarities in their brain lesions and/or in their brain lesions evolution, to identify and/or predict subpopulations that might benefit from a given treatment.

The present inventors have identified that state-of-the-art machine-learning methods for detecting brain lesions fail to capture the diversity of brain metastases, as they are trained on brain image train sets that are not representative of the high variability of the features that characterize brain metastases. In particular, train-test splits of the input datasets in state-of-the-art methods are randomly generated while not taking into account any characterizing feature of the metastases. In fact, state-of-the-art methods treat brain metastases as a single “whole tumor” class, or include necrosis at most, while brain metastases in MRI scans can have different types of appearance. Additionally, state-of-the-art methods do not consider the presence of the surgical cavity and/or edema regions to generate train-test splits.

The invention according to the present application discloses the surprising effect that by leveraging statistical distributions of the features annotated in the input dataset of brain digital images, it is possible to generate a train-test split, and therefore a train set, that is balanced and representative of all types of lesions. In particular, by increasing the similarity between said statistical distributions in the train sets and in the test sets, it is possible to reduce the potential bias in the train sets due to outliers of the features distributions, thus improving the ability of the models trained on such train sets to detect all types of lesions.

Thus, according to a first aspect, the present invention provides a computer-implemented method of generating a training dataset for a machine-learning model for brain lesion detection, the method comprising the steps of: receiving an input dataset of brain digital images, comprising annotated features; generating at least two train-test splits of the received input dataset, wherein each train-test split consists of a split of the input dataset in a pair of a train set and a test set; estimating, for the at least two generated train-test splits, one or more probability distributions associated with one or more of the annotated features in the train set and one or more probability distributions associated with one or more of the annotated features in the test set; calculating, for the at least two generated train-test splits and for the one or more annotated features, the divergence between the probability distributions estimated for the pair of the train set and the test set; aggregating, across the one of more annotated features, the calculated divergence for the at least two generated train-test splits; selecting the train-test split with the lowest aggregated divergence; outputting the train set of the selected train-test split. The method can further comprise outputting the test set of the selected train-test split. The method can further comprise outputting the selected train-test split.

The annotated features can comprise: number of enhancing tumor lesions, number of necrosis, number of edema regions, number of cavities, volume of enhancing tumor lesions, volume of necrosis, volume of edema regions, volume of cavities, and any combinations and/or aggregations thereof. In particular, the annotated features can comprise: the ratio between the total volume of necrosis and the total volume of combined necrosis and enhancing tumor lesions; and/or the total number of combined necrosis and enhancing tumor lesions; and/or the mean volume of the combined necrosis and enhancing tumor lesions; and/or the volume of the cavities;

and/or the volume of edema regions. Generating a train-test split with similar distributions of the ratio between the total volume of necrosis and the total volume of combined necrosis and enhancing tumor lesions allows to obtain improved models, trained on the train sets of such train-test splits, for the detection both of common brain metastases that appear as bright blobs and of less common brain metastases that appear as dark necrotic tissue on contrast-enhanced scans. Additionally, such improved trained models allow to identify as single metastases those brain metastases that contain both bright blobs and dark necrotic tissues, rather than as distinct metastases. Generating a train-test split with similar total number of combined necrosis and enhancing tumor lesions allows to obtain improved models, trained on the train sets of such train-test splits, for the detection of metastases in cases where only a handful of lesions are present and in cases where tens or hundreds of lesions are present. Generating a train-test split with similar distributions of the mean volume of the combined necrosis and enhancing tumor lesions allows to obtain improved models, trained on the train sets of such train-test splits, for the detection of small as well as big lesions. Generating a train-test split with similar distributions of the volume of the cavities and/or of the volume of the edema regions allows to obtain improved models, trained on the train sets of such train-test splits, for the detection of lesions in cases where surgical cavities and edema regions are present.

The input dataset can comprise brain digital images from MRI scans collected with sequences comprising: T1CE, T1, T1-FLAIR, T2, T2-FLAIR. By using a combination of MRI scans collected with different sequences, in particular the combination of T1CE, T1 or T1-FLAIR, T2, T2-FLAIR, as the input dataset of brain digital images, it is possible to annotate more lesions and/or the same lesions more precisely, thus improving the ability of the models trained on parts of such input datasets to detect more lesions and/or the same lesions more precisely.

The input dataset can comprise brain digital images of different subjects and/or brain digital images of the same subject collected at different points in time. The brain digital images can comprise the full brain of the subject. The brain digital images can comprise one or more portions of the brain of the subject. The subject can be a human subject. The subject can be an adult subject. The subject can be a paediatric subject. The subject can be a healthy subject. The subject can be a subject that has been diagnosed having a primary brain cancer or being likely to have a primary brain cancer. The subject can be a subject that has been diagnosed having a metastatic cancer or being likely to have a metastatic cancer. The subject can be a subject that has been diagnosed having a metastatic brain cancer or being likely to have a metastatic brain cancer. The brain digital images of the same subject collected at different points in time can have been collected during different visits. The brain digital images of the same subject collected at different points in time can have been collected during the same visit but during different scans. The brain digital images of the same subject collected at different points in time can have been collected during different visits and different scans. Different scans can comprise MRI scans with different MRI sequences. The brain digital images of different subjects can have been collected during different scans. Different scans can comprise MRI scans with different MRI sequences.

The invention according to the present application also discloses the surprising effect that by combining the statistical distributions of the features annotated per subject at the visit level with the statistical distributions of features annotated at the subject level, i.e. across visits, it is possible to generate a train-test split, and therefore a train set, that is balanced and representative of subjects with any number of visits. In particular, by constraining the train-test split to maintain desired proportions in terms of number of subjects as well as number of visits, it is possible to reduce the bias in the dataset towards subjects with a higher number of visits.

The train-test splits can be generated using the number of different subjects and/or the number of points in time at which brain digital images of the same subject are collected. In particular, the train-test splits can be generated by determining the desired proportions of the input dataset into the train set and the test set in terms of number of subjects. For example, the train set can comprise the brain digital images of 70% of the subjects in the input dataset and the test set can comprise the brain digital images of 30% of the subjects in the input dataset. Alternatively, the train-test splits can be generated by determining the desired proportions of the input dataset into train set and test set in terms of number of visits of the subjects. For example, the train set can comprise the brain digital images from 70% of the visits in the input dataset and the test set can comprise the brain digital images from 30% of the visits in the input dataset. The train-test splits can be generated by determining the desired proportions of the input dataset into the train set and the test set in terms of a combination of the number of subjects and the number of visits. The train-test splits can be randomly generated. For example, 100000 train-test splits can be randomly generated. The train-test splits can be randomly generated with determined desired proportions of the input dataset into the train set and the test set. The train-test splits can be randomly generated with predetermined desired proportions of the input dataset into the train set and the test set.

The method can have one or more of the following features.

The step of estimating, for the at least two generated train-test splits, one or more probability distributions associated with one or more of the annotated features in the train set and one or more probability distributions associated with one or more of the annotated features in the test set can comprise performing kernel density estimation methods in the train set and in the test set in the space of the one or more annotated features.

The step of calculating, for the at least two generated train-test splits and for the one or more annotated features, the divergence between the probability distributions estimated for the pair of the train set and the test set can comprise calculating, for the at least two generated train-test splits and for the one or more annotated features, the similarity between the probability distributions estimated for the pair of the train set and the test set; and the step of aggregating, across the one or more annotated features, the calculated divergence for the at least two generated train-test splits can comprise aggregating, across the one or more annotated features, the calculated similarity for the at least two generated train-test splits; and the step of selecting the train-test split with the lowest aggregated divergence can comprise selecting the train-test split with the highest aggregated similarity. The step of calculating the divergence can comprise calculating the Jensen-Shannon divergence. The step of aggregating the calculated divergence for the at least two generated train-test splits can comprise calculating any summarized metrics of the divergence across the one or more annotated features. Summarized metrics can comprise mean, median, trimmed versions thereof. The step of calculating the similarity can comprise performing one or more similarity measurements. For example, similarity measurements can comprise cosine similarity measurement, Intersection over Unit measurements, statistical tests, in particular p-value tests, or combinations thereof. The step of aggregating the calculated similarity for the at least two generated train-test splits can comprise calculating any summarized metrics of the similarity across the one or more annotated features. Summarized metrics can comprise mean, median, trimmed versions thereof.

According to a second aspect, the present invention provides a method of training one or more machine-learning models for brain lesion detection, comprising the steps of: receiving an input training dataset generated according to the first aspect; training one or more machine-learning models using the received input training dataset; (optionally) outputting the trained one or more machine-learning models.

The method according to the second aspect can further comprise the step of assessing the performance of the trained one or more machine-learning models, wherein assessing the performance comprises evaluating true positives, false positives, false negatives, and combinations and/or aggregations thereof, in particular JaccardIndex, precision, recall. True positives consist of detected lesions matching annotated lesions. False positives consist of detected lesions not matching annotated lesions. False negatives consists of non-detected annotated lesions. The method can further comprise the step of selecting at least one of the one or more trained machine-learning model using the assessed performance. For example, the one model with the highest precision can be selected. Alternatively, the two models with the highest precision can be selected. Alternatively, the one model with the highest recall can be selected. Alternatively, the one model with the highest average between precision and recall can be selected. Further alternatives are implicitly disclosed as immediate to the skilled in the art.

The machine-learning models trained according to the second aspect are able to detect brain lesions, and in particular brain metastases, with different appearances in the MRI scans: bright blobs or necrotic areas, small or big, few or many. The machine-learning models trained according to the second aspect are also able to identify lesions comprising both bright blobs and necrotic areas as single lesions rather than distinct lesions. The machine-learning models trained according to the second aspect are also able to detect brain lesions, and in particular brain metastases, in the presence of surgical cavities and/or edema regions. The machine-learning models trained according to the second aspect do not suffer from a bias when trained on train sets comprising brain digital images from the same patients at different points in time. In other words, the machine-learning models trained according to the second aspect do not learn the specificities of brain metastases of a particular subject even if trained with multiple images, i.e. multiple scans from different visits, of the same subject.

Thus, according to a third aspect, the present invention provides a method of using one or more machine-learning models, trained according to the second aspect, to detect brain lesions, the method comprising the steps of: receiving an input brain digital image, detecting brain lesions in the received input image, (optionally) outputting the detected brain lesions. The method can further comprise extracting features of the detected brain lesions, and optionally outputting the extracted features. The step of detecting brain lesions can further comprise the step of segmenting brain lesions prior to detection. The one or more machine-learning models can comprise a first model and a second model, and outputting the detected brain lesions can comprise obtaining first brain lesions detected by the first model, obtaining second brain lesions detected by the second model, and merging the first brain lesions and second brain lesions. The first model can be a nnDetection model and the second model can be a nnUNet model. Merging the first brain lesions and second brain lesions can comprise performing one or more of the following set operations between the two sets of lesions: intersection, union, left join, right join. According to a fourth aspect, the present invention provides a method of monitoring and/or predicting brain lesions in a subject, the method comprising using the computer-implemented method of any of the preceding claims, in particular wherein the subject is undergoing or has undergone a treatment. For example, the method according to the third aspect can be used on a first input brain digital image and on a second brain digital image of a subject, wherein the first image has been collected before a treatment and the second image has been collected during treatment. Alternatively, the first image can have been collected during treatment and the second image can have been collected after treatment. Alternatively, the first image can have been collected before treatment and the second image can have been collected after treatment. Alternatively, the first image can have been collected during a first treatment and the second image can have been collected during a second treatment. Further alternatives are implicitly disclosed as immediate to the skilled in the art. Similarly, the method according to the third aspect can be used in the described manner on more than two brain digital images.

According to a further aspect, there is provided a computer program [product] comprising instructions which, when the program is executed by a computer, cause the computer to carry out the steps of the method of any preceding aspects.

According to a further aspect, there is provided a system comprising: a processor; and a computer readable medium comprising instructions that, when executed by the processor, cause the processor to perform the steps of the method of any preceding aspects; optionally a brain digital image acquisition means.

### BRIEF DESCRIPTION OF THE FIGURES

FIG. 1 illustrates an embodiment of a system that can be used to implement one or more aspects described herein.

FIG. 2 is a flow diagram showing, in schematic form, a method of generating a training dataset for a machine-learning model for brain lesion detection, according to the invention.

FIG. 3 is a flow diagram showing, in schematic form, a method of training one or more machine-learning models for brain lesion detection with a training dataset generated according to FIG. 2, according to the invention.

FIG. 4 is a flow diagram showing, in schematic form, a method of using one or more machine-learning models, trained according to FIG. 3, to detect brain lesions, according to the invention.

FIG. 5 shows different methods of performing lesion measurements in oncology.

FIG. 6 shows an example of generated 3D reconstruction from orthogonal views of three 2D scans.

FIG. 7 shows an example of annotated subregions (active tumor and surgical cavity).

FIG. 8 shows the probability distributions of the stratification variables in the train set and in the test set.

FIG. 9 shows the histogram of true positives as obtained from nnUNet and nnDetection.

FIG. 10 shows the confusion matrices summarizing the comparison between ground truth and prediction for the total volume parameter (left) and the blob count parameter (right).

### DETAILED DESCRIPTION

In describing the present invention, the following terms will be employed, and are intended to be defined as indicated below.

As used herein “data” and “images” are used interchangeably unless otherwise specified.

As used herein machine-learning “algorithms” and “models” are used interchangeably unless otherwise specified.

As used herein and unless otherwise specified “training dataset” or “training set” or “train set” is a dataset used for training models, the dataset comprising digital images. As used herein and unless otherwise specified “testing dataset” or “testing set” or “test set” is a dataset used for testing trained models, comprising digital images not used to train said models. As used herein and unless otherwise specified “validation dataset” or “validation dataset” or “validation set” is a dataset used for validating trained models, comprising digital images not used to train or test said models.

As used herein “training” a machine-learning model assumes the standard meaning known to the person skilled in the art, and comprises finding the best combination of model parameters, e.g. weights and biases (depending on the architecture of the model), to minimize a loss function over training data. As used herein “ground truth” corresponds to annotated lesions.

As used herein “segmenting” objects of interest assumes the technical meaning of identifying contours (e.g. 2D bounding boxes, 3D bounding boxes) around the objects of interest. The “segmentation metrics” measure the overlap between such contours. As used herein “detecting” segmented objects of interest assumes the technical meaning of finding separate instances of the segmented objects, and/or characterizing such separate instances, e.g. obtaining their location (coordinates, relative distances), their number.

As used herein the term “sequence” has the specific technical meaning of a magnetic resonance image collected with a particular setting of pulse sequences and pulse field gradient, resulting in a particular image appearance defined by the gray levels in which different tissues appear. In particular, several sequences are explicitly claimed in the present invention: T1CE, T1, T1-FLAIR, T2, T2-FLAIR. T1CE stands for T1 Contrast Enhanced, also known as post-contrast. T1CE are sequences performed after the infusion of a contrast enhancement agent, like for example Gadolinium. These sequences differ in terms of Repetition Time (TR) and Time to Echo (TE) with which the images are created: TR is the amount of time between successive pulse sequences applied to the same slide; TE is the time between the delivery of the Radio Frequency (RF) pulse and the receipt of the echo signal. T1 sequences are produced by using short TE, preferably chosen to be approximately 14 milliseconds, and short TR, preferably chosen to be approximately 500 milliseconds. T2 sequences are produced by using longer TE, preferably chosen to be approximately 90 milliseconds, and longer TR, preferably chosen to be approximately 4000 milliseconds. FLAIR stands for Fluid Attenuated Inversion Recovery. Further details about the MRI sequences mentioned explicitly or not in the present application are considered common knowledge of the person skilled in the art.

As used herein the term “enhancing tumor lesions” or “active tumor lesions” comprises tumor lesions that appear with enhanced intensity due to for example the contrast enhancement agent used, or the specificities of the sequences used.

As used herein and unless otherwise specified the term “stratification factors” or “stratification variables” comprise annotated features, and/or characterizing features or parameters, used to stratify the dataset, e.g. to divide the dataset into data subsets.

The systems and method described herein can be implemented in a computer system, in addition to the structural components and user interactions described. As used herein, the term “computer system” includes the hardware, software and data storage devices for embodying a system and carrying out a method according to the described embodiments. For example, a computer system can comprise one or more central processing units (CPU) and/or graphics processing units (GPU), input means, output means and data storage, which can be embodied as one or more connected computing devices. Preferably the computer system has a display or comprises a computing device that has a display to provide a visual output display. The data storage can comprise RAM, disk drives, solid-state disks or other computer readable media. The computer system can comprise a plurality of computing devices connected by a network and able to communicate with each other over that network. It is explicitly envisaged that computer system can consist of or comprise a cloud computer.

The methods described herein are computer implemented unless context indicates otherwise.

### Systems

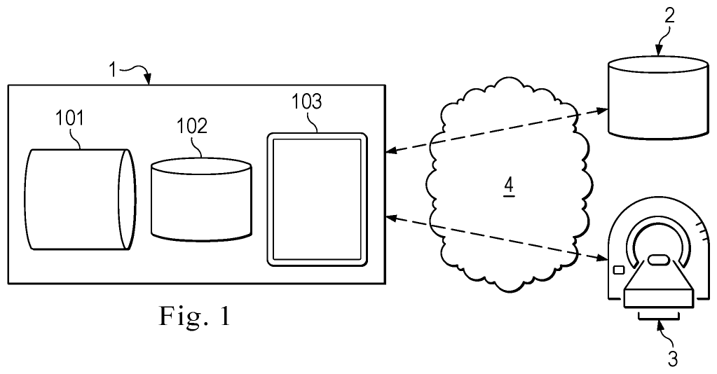

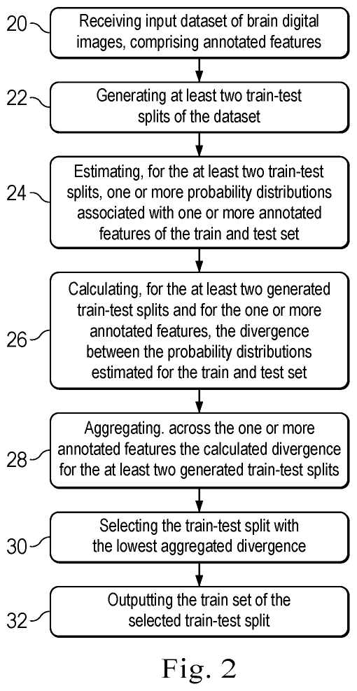

FIG. 1 illustrates an embodiment of a system that can be used to implement one or more aspects described herein. With reference to FIG. 1, the system comprises a computing device 1, which comprises a processor 101 and a computer readable memory 102. In the embodiment shown, the computing device 1 also comprises a user interface 103, which is illustrated as a screen but can include any other means of conveying information to a user such as e.g. through audible or visual signals. The computing device 1 is communicably connected, such as e.g. through a network, to MRI images acquisition means 3, such as an MRI scanner, and/or to one or more databases 2 storing MRI images. The one or more databases 2 can further store one or more of: control data, parameters (such as e.g. thresholds derived from control data, parameters used for normalization, etc.), clinical and/or patient related information, etc. The computing device can be a smartphone, tablet, personal computer or other computing device. The computing device can be configured to implement a method of processing MRI images, as described herein. In alternative embodiments, the computing device 1 is configured to communicate with a remote computing device (not shown), which is itself configured to implement a method of generating a training dataset for a machine-learning model for brain lesion detection, as described herein. In such cases, the remote computing device can also be configured to send the result of the method of generating a training dataset for a machine-learning model for brain lesion detection. Communication between the computing device 1 and the remote computing device can be through a wired or wireless connection, and can occur over a local or public network 4 such as e.g. over the public internet. The MRI images acquisition means 3 can be in wired connection with the computing device 1, or can be able to communicate through a wireless connection, such as e.g. through WiFi and/or over the public internet, as illustrated. The connection between the computing device 1 and the MRI images acquisition means 3 can be direct or indirect (such as e.g. through a remote computer). The MRI images acquisition means 3 are configured to generate a training dataset for a machine-learning model for brain lesion detection. In some embodiments, the MRI images can have been subject to one or more preprocessing steps (eg cropping, resizing, normalizing, registration, coregistration, etc) prior to performing the methods described herein.

### Methods

FIG. 2 is a flow diagram showing, in schematic form, a method of generating a training dataset for a machine-learning model for brain lesion detection, according to the invention. With reference to FIG. 2, at step 20 an input dataset of brain digital images comprising annotated features are received. At step 22 at least two train-test splits of the input dataset are generated. Train-test splits are splits of the dataset in pairs of train set and test set. At step 24, for the at least two generated train-test splits, one or more probability distributions associated with one or more of the annotated features in the train set are estimated, and one or more probability distributions associated with one or more of the annotated features in the test set are estimated. At step 26, for the at least two generated train-test splits and for the one or more annotated features, the divergence between the probability distributions estimated for the pair of the train set and the test set are calculated. At step 28 the calculated divergence for the at least two generated train-test splits are aggregated across the one or more annotated features. At step 30 the train-test split with the lowest aggregated divergence is selected. At step 32 the train set of the selected train-test split is outputted.

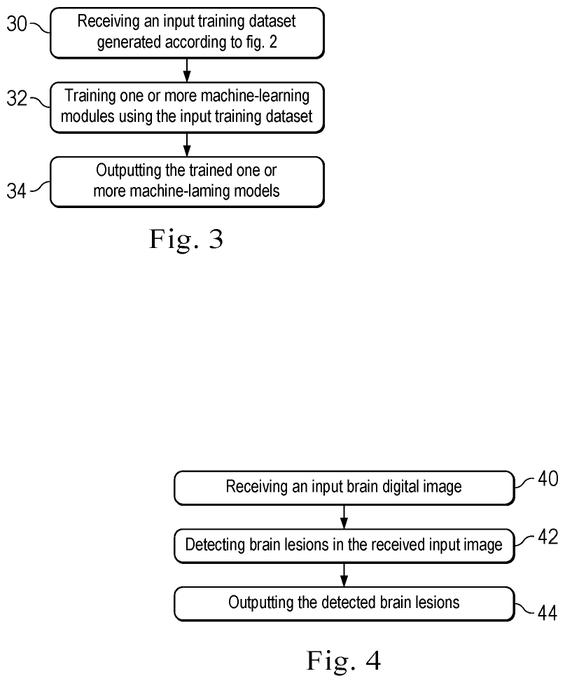

FIG. 3 is a flow diagram showing, in schematic form, a method of training one or more machine-learning models for brain lesion detection with a training dataset generated according to FIG. 2, according to the invention. With reference to FIG. 3, at step 30 an input training dataset generated according to FIG. 2 is received. At step 32, one or more machine-learning models are trained using the received input training dataset. At step 34, the trained one or more machine-learning models are outputted.

FIG. 4 is a flow diagram showing, in schematic form, a method of using one or more machine-learning models, trained according to FIG. 3, to detect brain lesions, according to the invention.

With reference to FIG. 4, at step 40 an input brain digital image is received. At step 42, brain lesions in the received input image are detected. At step 44, the detected brain lesions are outputted.

### EXAMPLES

The examples below illustrate applications of the methods of the present invention.

### Example 1—Generating a Training Dataset for Brain Metastases Detection

In this example, data acquired during a randomized, multicenter, phase III study of Alectinib versus Crizotonib was used. The study was focused on the treatment of treatment-naïve anaplastic lymphoma kinase-positive advanced NSCLC (Non-Small Cell Lung Cancer)¹¹.

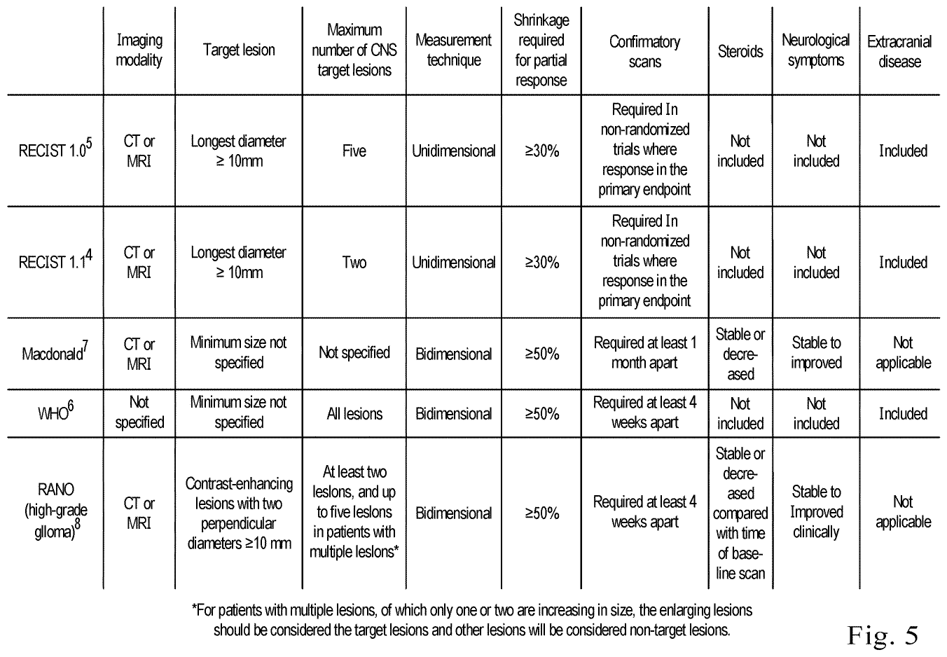

The data from the clinical study contained multiple visits for most of the patients, so that it was possible to track the progression of the disease. The lesions were divided into two types: target and non-target lesions, based on Response Assessment in Neuro-Oncology (RANO) criteria, according to FIG. 5. Target lesions were counted, measured, and reported. The lesion measurements were saved as DICOM-RT files. The consecutive visits were analyzed as time series to assess the response to the treatment. As target lesions in RANO, contrast-enhancing lesions with the sum of two perpendicular diameters equal to or larger than 10 mm were considered.

In the clinical study, the patients were divided into two groups: 1) patients with target lesions present on baseline scans; 2) patients with no target lesions present on baseline scans. Baseline scans are scans performed on the first of a series of visits. Patients in the second group usually developed brain metastases at some point in the trial. Such lesions were measured by radiologists in the scope of the clinical study, but were marked as non-target lesions and did not contribute to overall RANO scores.

Ultimately, 275 studies from 87 patients were annotated. These studies were obtained from 53 different sites. The statistics of selected patients is shown in Table I.

Of these selected patients, only studies with the following MRI sequences available were retained: T1CE, T1/T1-FLAIR, T2, T2-FLAIR. T1-FLAIR sequences were used interchangeably with T1 sequences since outputs of these two protocols only differ for the contrast. T1CE sequences had to be acquired in high-quality 3D protocol. The statistics of slice thickness values for different MRI sequences is shown in Table II.

Additionally, a study selection based on the lesion measurements obtained during the clinical trials visits was further performed to select the most representative and diverse subset of patients. The measurements were analyzed to track the changes in lesion appearances and used in the process of series selection. Images showing no changes in lesion appearances in two consecutive visits were not used in the training dataset, to avoid that the model would memorize such images. The following approach to study selection was used for the two groups of patients above mentioned: 1) for patients with target lesions on baseline scans, available measurements from all visits were used; 2) for patients with no target lesions on baseline scans, the measurements of the following visits were used: first visit, last visit, and all visits for which the lesion changes. In case of constant progression or regression for a couple of visits, only first and last visits were used. The number of studies selected was 290.

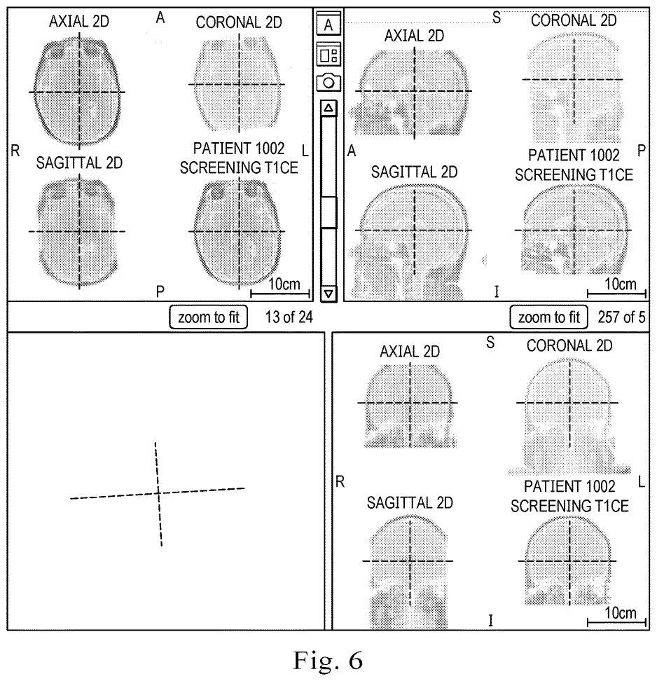

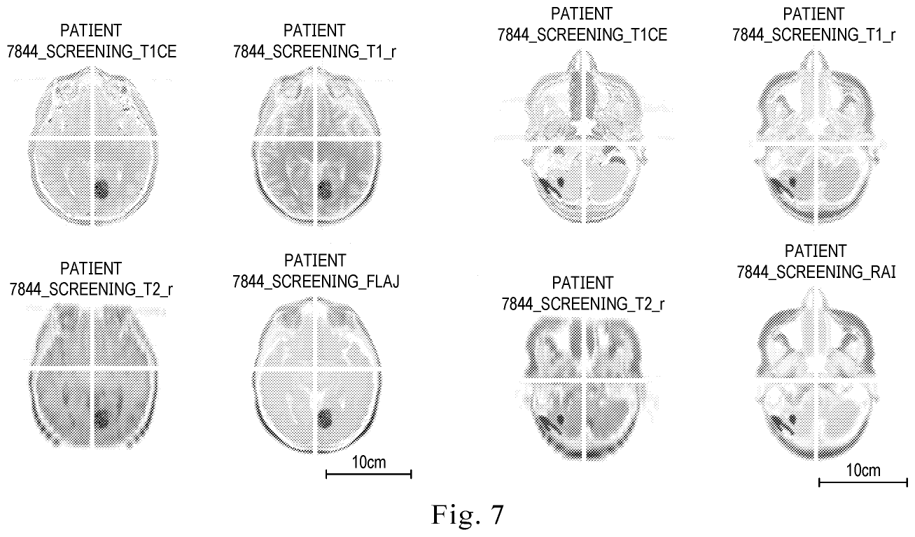

For 133 visits, no 3D T1CE sequences were available, and three 3D orthogonal thick series were obtained instead. These series were generated artificially from the original 3D series to reduce the memory footprint of the scans. To recreate missing spatial information for such studies, NiftyMIC (a tool for 3D reconstruction of isotropic, high-resolution volumes from multiple stacks of low-resolution 2D slices) with default parametrization was used. An example of this process is shown in FIG. 6.

The data were annotated by two expert radiologists (22 and 20 years of experience respectively) and five experienced raters (8, 8, 7, 5 and 4 years of experience). The former ones were responsible for reviewing the annotations prepared by the latter ones. Each study annotation required acceptance from at least one expert radiologist, whereas the annotations of baselines studies (first visits of each patient) were double-checked by both experts. Each study contained a T1CE high-quality series. Such series were used as reference scans for the annotation process. The doctors were tasked with contouring the following four subregions in the images: enhancing tumor, necrosis, edema, surgical cavity. An example of annotated subregions is shown in FIG. 7: a single active lesion is shown (left) and a surgical cavity (right). The doctors specified their confidence levels (1-4) for each class separately (along with one additional confidence value for overall segmentation) for every annotation. If they were able to identify voxels of a class and were confident about the output segmentation or if they were confident that a specific class was missing from the image, they marked it with confidence level 4. As the experts were confident about the annotations, the confidence levels were not incorporated in the analysis of the data. Doctors were asked to assess the quality of each scan by assigning a binary value to each study (acceptable vs unacceptable to diagnostic purposes). This step was crucial to make sure that MRI data with reconstruction artifacts were excluded from the analysis. After this quality assessments, 15 scans were removed from the dataset. The final dataset contained 275 studies from 87 patients. The statistics of total volumes of the annotations are presented in Table III. The statistics of the number of visits across patients is shown in Table IV.

Annotated images were preprocessed, wherein the preprocessing comprised brain extraction. For some skull-stripped studies, annotated voxels were shown outside the brain masks. As these could mislead the model, all such voxels were removed and the annotations were trimmed to the brain masks. All such cases were analyzed, some manually, to confirm that all removed voxels consisted of areas of surgical cavity or edema.

The data presented extremely complex data characteristics: the dataset was heterogeneous, there were outliers, necrosis, many small bright blobs as well as huge single blobs. To ensure that all different types of lesions were represented in both the training set and the test set, the following stratification factors were defined:
Ratio of total volume of necrosis and total volume of necrosis plus enhancing tumor (nec_to_et+nec);
Enhancing tumor plus necrosis blob count (et_nec_blob_count);
Enhancing tumor plus necrosis total volume (et_nec_mean_volume);
Cavity total volume (cav_tot_volume);
Edema total volume (ed_tot_volume).

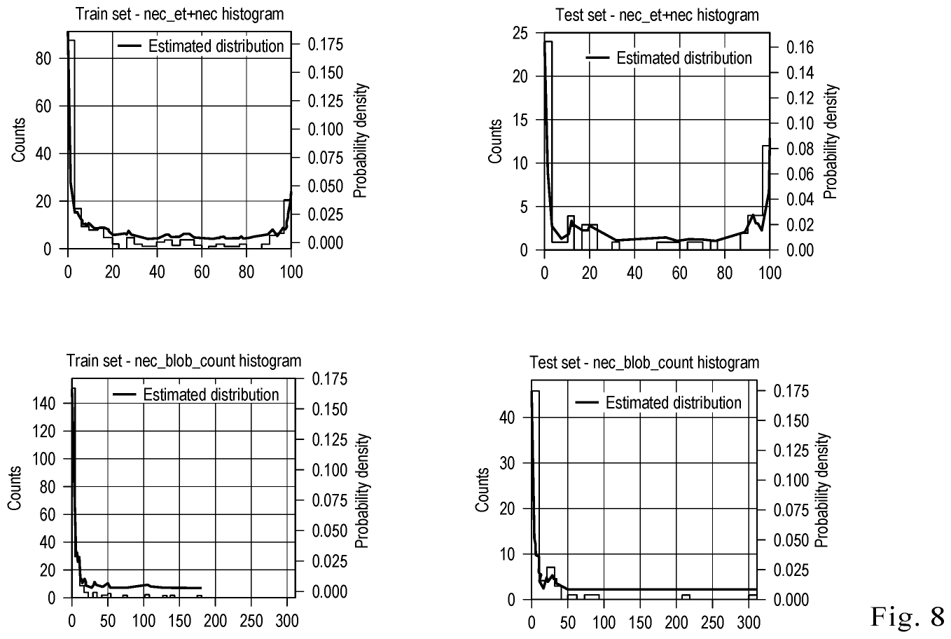

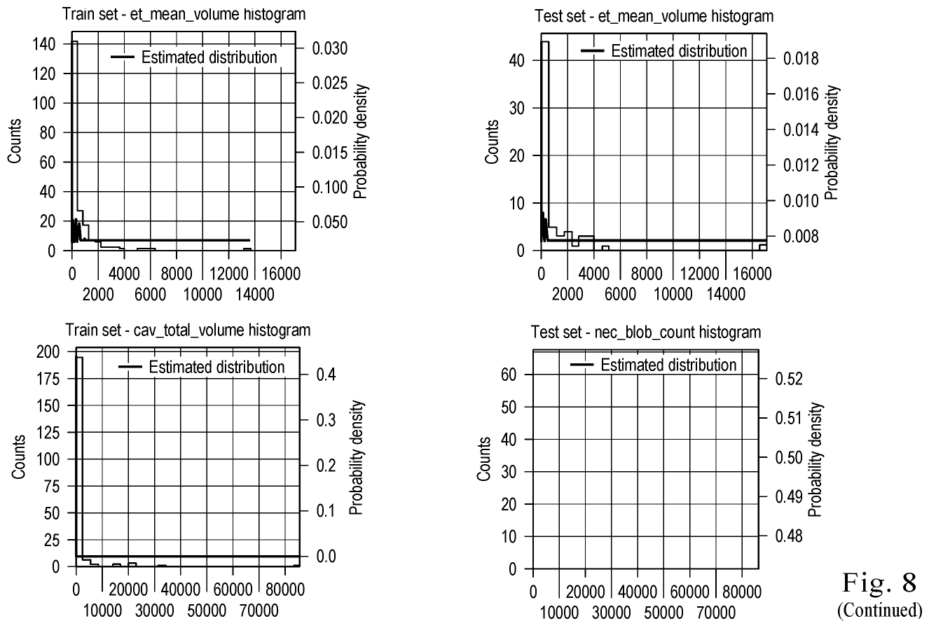

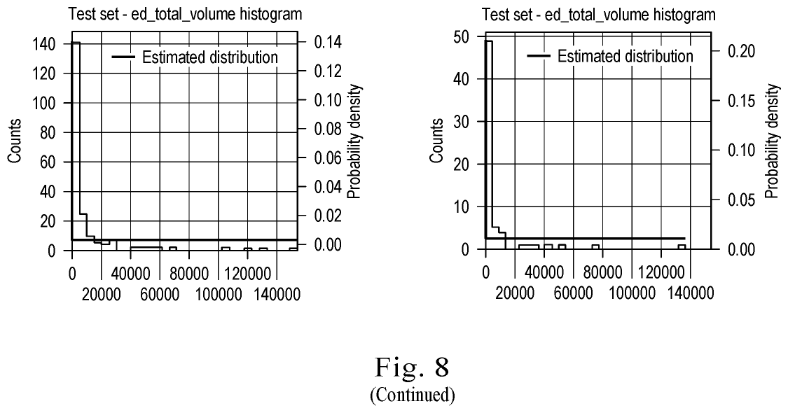

The visit-based statistics of all calculated features are presented in Table V, and show how diverse the values were, especially on the extreme ends of the ranges. Due to the many outliers, the standard stratification approach of creating subsets containing permutations of the features was not possible. In view of the data characteristics, the stratification method employed uses histogram matching of feature distributions of the patients. To ensure that there is no training-test information leak at the patient level (across visits), the sets were partitioned at the patient level. However, the stratification factors listed above operate at the visit level. Therefore the following stratification steps were performed:
1. Generate representative samples of train-test splits using the number of visits for each patient as the only stratification factor. This resulted in maintaining the desired proportions (70%-30%) not only for the number of patients but also for the total number of visits. 100000 candidate splits were randomly generated.
2. Estimate probability distributions for each train and test set using kernel density estimation methods in the space of other stratification variables. The feature distributions for the 5 above listed stratification variables are shown in FIG. 8.
3. Calculate Jensen-Shannon divergence between each pair of train and test set.
4. Choose the pair with the lowest divergence between estimated probability distributions.

The final split into training set and test set was as follows: 1) Training set: 210 visits for 68 patients; 2) Test set: 65 visits for 18 patients. The distribution of number of visits for the patients in the train set and in the test set are shown in Table VI.

A similar approach was used to generate validation folds from the training set. A representative sample of 5-fold splits was generated, with 100000 candidate splits. The distributions for each of them were estimated. The scoring method was performed in the following way:
1. For a given validation split candidate, calculate the Jensen-Shannon divergence for each pair of folds resulting in 10 divergence values (2-element combinations from the set of 5 folds);
2. Average the divergence values;
3. Choose the validation split candidate with the lowest average divergence value.

### Example 2—Training DNN Models for Brain Metastases Detection

In this example, different DNN architectures were employed. The models were trained on two MRI sequence configurations: T1CE only and all 4 modalities available. All experiments were run for a single class created by merging the two classes of enhancing tumor and necrosis. Outputs of all experiments were bounding boxes. In the end, the combination of the best performing models was selected and assembled into the final processing pipeline.

All networks were trained on voxel-based segmentations as input data.

The nnDetection is a self-configuring framework for 3D (volumetric) medical object detection which can be applied to new datasets without manual intervention. The nnDetection was run with default parameters. All nnDetection models were trained with cross-validation on custom 5-fold splits, as there is not off-the-shelf option to run a training on all data in the nnDetection framework.

The nnUNet is a robust and self-adapting framework for a U-Net based medical image segmentation. The nnUNet was run with default parameters. Due to the limited amount of data, a single model was trained on the whole training dataset. Preliminary trainings with cross-validation were performing significantly worse and were not pursued further.

The DeepMedic+ is a deep learning framework for brain metastases detection and segmentation in longitudinal MRI data. The DeepMedic+ framework was run not only with default parameters, but also with different values of the alpha parameter, a weighting factor in the loss function, designed by the authors to balance precision and recall. The lower the value of alpha, the higher the precision of the model. Two different approaches were investigated: 1) prediction on a single study; 2) predictions on two consecutive studies. DeepMedic+ models were trained using T1CE only since the framework supported one channel from a single time point in default configuration.

The basic steps of the processing pipeline, both for training and prediction, were as follows:
Registration to T1CE: a greedy registration tool was used. Rigid transformation on skull-stripped scans, with 20 millimeter dilation of brain masks was performed. T1CE series were used as a high-quality reference series for ground-truth preparation, so they were used as reference input for the registration algorithm as well.
Brain extraction: HD-BET tool was used with default parametrization. All MRI sequences were skull-stripped separately, then masks were merged by calculating the union.
Model training/Inference.
Segmentation/detection to bounding boxes on a single model or a couple of models by merging prediction from multiple sources.

For DeepMedic+ the co-registration was performed in time: each reference T1CE series of a previous study was registered to the T1CE series of the following one.

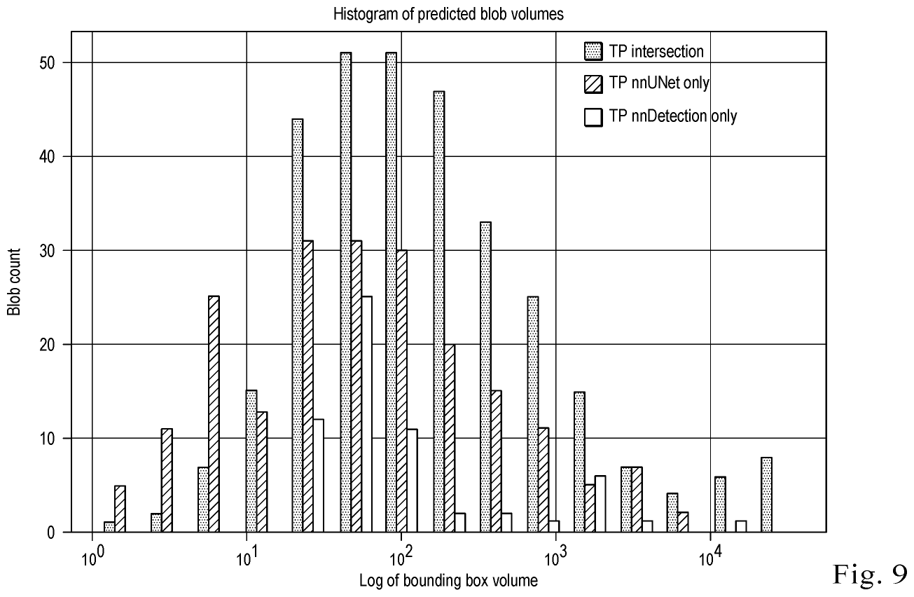

Predictions from the different models were analyzed, with the results that detection-based and segmentation-based architectures tend to see different lesions. Since nnUNet was trained on voxel-based granularity, it was able to localize much smaller objects than nnDetection, but missed some bigger blobs detected by the latter. FIG. 9 shows the histogram of true positives as obtained from nnUNet and nnDetection: blue-lesions matched for both models, green-lesions detected by nnUNet only, red-lesions detected by nnDetection only).

Thus, the two models were merged in the final model. The prediction merging algorithm was designed to combine overlapping bounding boxes generated by segmentation- and detection-based models. The algorithm worked as follows:
1. All blobs from the two/more sources were matched one-to-one with each other based on the highest overlap. For each pair of matched blobs, the bigger blob was added to the output (the other was considered to be redundant).
2. If there were any other blobs overlapping the matched blobs, they were removed as well to limit the number of potential false positives.
3. All blobs that do not overlap with any others were returned as separate hits.

Since nnUNet and DeepMedic+ provided voxel-based segmentation as output, while nnDetection generated 3D bounding boxes indicating the location and size of the lesions, to compare these approaches bounding boxes were generated from the segmentations by calculating the smallest cube that fits each of them. The study focused on instance-based detection metrics. Such metrics reflected how many lesions were localized correctly regardless of their size. These metrics help assess the performance of models in terms of blob identification, especially for patients with many small lesions. Detection metrics allow to track the number of lesions. To analyze their size, volumetric metrics were generated as well.

In order to calculate the metrics, the following steps were performed:
1. Extract all separate 3D blobs (representing tumors) from ground truth and prediction masks.
2. Calculate the number of true positives (TP), false positives (FP), and false negatives (FN) between blobs. The ground truth and predicted blobs were matched one by one:
   a. TP was counted if a ground truth blob overlapped with a prediction blob by a minimum Intersection over Unit (IoU) of 0.1. If there were many blobs overlapping, the matched pair of blobs was determined based on the highest overlap of all pairs.
   b. FN was counted when there was no blob matched with a ground truth blob.
   c. FP was counted when there was no blob matched with a prediction blob.
3. Use the TP, FP and FN to calculate JaccardIndex, Precision and Recall as follows:

$$JaccardIndex = \frac{T P}{T P + F P + F N}$$

$$Precision = \frac{T P}{T P + F P}$$

$$Recall = \frac{T P}{T P + F N}$$

In the study, two sets of statistics for all experiments were calculated, due to the high variability in the number of lesions and number of visits for different patients:
Visit-based: metrics were calculated for each visit separately and then aggregated.
Blob-based: metrics were calculated for all blobs for all patients, no aggregation needed.

To select the final model, the balance between the visit-based and blob-based performance was maintained. Since nnUNet-like architectures performed best for separate blobs, and nnDetection performed best for separate visits, these two architectures were combined in the final model. Quantifying the performance of detection algorithms is particularly challenging when it comes to small objects, as they are hard to localize but at the same time rarely have a notable impact on the overlap metrics. With the two types of metrics described above, the best model was selected in terms of detection and tracking of brain metastases.

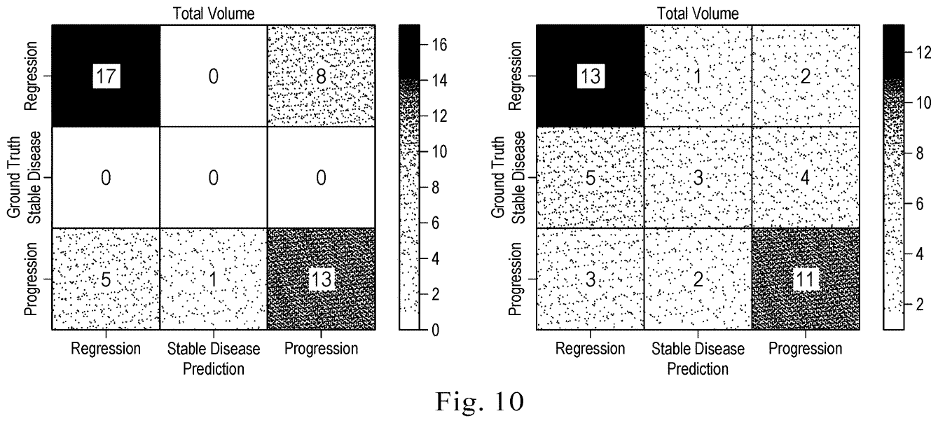

The selected final model performed very well in terms of predicting disease progression. FIG. 10 shows the confusion matrices summarizing the comparison between ground truth and prediction for the total volume parameter (left) and the blob count parameter (right).

### EMBODIMENTS

1. In an embodiment, a computer-implemented method of generating a training dataset for a machine-learning model for brain lesion detection is disclosed, the method comprising the steps of:
   a. receiving an input dataset of brain digital images, comprising annotated features;
   b. generating at least two train-test splits of the received input dataset, wherein each train-test split consists of a split of the dataset in a pair of a train set and a test set;
   c. estimating, for the at least two generated train-test splits, one or more probability distributions associated with one or more of the annotated features in the train set and one or more probability distributions associated with one or more of the annotated features in the test set;
   d. calculating, for the at least two generated train-test splits and for the one or more annotated features, the divergence between the probability distributions estimated for the pair of the train set and the test set;
   e. aggregating, across the one or more annotated features, the calculated divergence for the at least two generated train-test splits;
   f. selecting the train-test split with the lowest aggregated divergence;
   g. outputting the train set of the selected train-test split.
2. In an embodiment, a computer-implemented method of generating a training dataset for a machine-learning model for brain lesion detection is disclosed, the method comprising the steps of:
   a. receiving an input dataset of brain digital images, comprising annotated features;
   b. generating at least two train-test splits of the received input dataset, wherein each train-test split consists of a split of the dataset in a pair of a train set and a test set;
   c. estimating, for the at least two generated train-test splits, one or more probability distributions associated with one or more of the annotated features in the train set and one or more probability distributions associated with one or more of the annotated features in the test set;
   d. calculating, for the at least two generated train-test splits and for the one or more annotated features, the divergence between the probability distributions estimated for the pair of the train set and the test set;
   e. aggregating, across the one or more annotated features, the calculated divergence for the at least two generated train-test splits;
   f. selecting the train-test split with the lowest aggregated divergence;
   g. outputting the train set of the selected train-test split.
   h. outputting the test set of the selected train-test split.
3. In an embodiment, a computer-implemented method of generating a training dataset for a machine-learning model for brain lesion detection is disclosed, the method consisting of the steps of:
   a. receiving an input dataset of brain digital images, comprising annotated features;
   b. generating at least two train-test splits of the received input dataset, wherein each train-test split consists of a split of the dataset in a pair of a train set and a test set;
   c. estimating, for the at least two generated train-test splits, one or more probability distributions associated with one or more of the annotated features in the train set and one or more probability distributions associated with one or more of the annotated features in the test set;
   d. calculating, for the at least two generated train-test splits and for the one or more annotated features, the divergence between the probability distributions estimated for the pair of the train set and the test set;
   e. aggregating, across the one or more annotated features, the calculated divergence for the at least two generated train-test splits;
   f. selecting the train-test split with the lowest aggregated divergence;
   g. outputting the train set of the selected train-test split.
4. In an embodiment, a computer-implemented method of generating a training dataset for a machine-learning model for brain lesion detection is disclosed, the method consisting of the steps of:
   a. receiving an input dataset of brain digital images, comprising annotated features;
   b. generating at least two train-test splits of the received input dataset, wherein each train-test split consists of a split of the dataset in a pair of a train set and a test set;
   c. estimating, for the at least two generated train-test splits, one or more probability distributions associated with one or more of the annotated features in the train set and one or more probability distributions associated with one or more of the annotated features in the test set;
   d. calculating, for the at least two generated train-test splits and for the one or more annotated features, the divergence between the probability distributions estimated for the pair of the train set and the test set;
   e. aggregating, across the one or more annotated features, the calculated divergence for the at least two generated train-test splits;
   f. selecting the train-test split with the lowest aggregated divergence;
   g. outputting the train set of the selected train-test split.
   h. outputting the test set of the selected train-test split.
5. In an embodiment, the method of any preceding embodiments is disclosed, wherein the annotated features comprise: number of enhancing tumor lesions, number of necrosis, number of edema regions, number of cavities, volume of enhancing tumor lesions, volume of necrosis, volume of edema regions, volume of cavities, and any combinations and/or aggregations thereof.
6. In an embodiment, the method of any preceding embodiments is disclosed, wherein the annotated features comprise: number of enhancing tumor lesions, number of necrosis, number of edema regions, number of cavities, volume of enhancing tumor lesions, volume of necrosis, volume of edema regions, volume of cavities, or any combinations and/or aggregations thereof.
7. In an embodiment, the method of any preceding embodiments is disclosed, wherein the annotated features comprise: number of enhancing tumor lesions, number of necrosis, number of edema regions, number of cavities, volume of enhancing tumor lesions, volume of necrosis, volume of edema regions, volume of cavities, and any combinations and/aggregations thereof.
8. In an embodiment, the method of any preceding embodiments is disclosed, wherein the annotated features comprise: number of enhancing tumor lesions, number of necrosis, number of edema regions, number of cavities, volume of enhancing tumor lesions, volume of necrosis, volume of edema regions, volume of cavities, and any combinations or aggregations thereof.
9. In an embodiment, the method of any preceding embodiments is disclosed, wherein the annotated features comprise: number of enhancing tumor lesions, number of necrosis, number of edema regions, number of cavities, volume of enhancing tumor lesions, volume of necrosis, volume of edema regions, volume of cavities, or any combinations and aggregations thereof.
10. In an embodiment, the method of any preceding embodiments is disclosed, wherein the annotated features comprise: number of enhancing tumor lesions, number of necrosis, number of edema regions, number of cavities, volume of enhancing tumor lesions, volume of necrosis, volume of edema regions, volume of cavities, or any combinations or aggregations thereof.
11. In an embodiment, the method of any preceding embodiments is disclosed, wherein the input dataset comprises brain digital images from MRI scans collected with sequences comprising: T1CE, T1, T1-FLAIR, T2, T2-FLAIR.
12. In an embodiment, the method of any preceding embodiments is disclosed, wherein the input dataset comprises brain digital images from MRI scans collected with sequences comprising: T1CE, T1, T2, T2-FLAIR.
13. In an embodiment, the method of any preceding embodiments is disclosed, wherein the input dataset comprises brain digital images from MRI scans collected with sequences comprising: T1CE, T1-FLAIR, T2, T2-FLAIR.
14. In an embodiment, the method of any preceding embodiments is disclosed, wherein the input dataset comprises brain digital images of different subjects and/or brain digital images of the same subject collected at different points in time.
15. In an embodiment, the method of any preceding embodiments is disclosed, wherein the input dataset comprises brain digital images of different subjects and brain digital images of the same subject collected at different points in time.
16. In an embodiment, the method of any preceding embodiments is disclosed, wherein the input dataset comprises brain digital images of different subjects or brain digital images of the same subject collected at different points in time.
17. In an embodiment, the method of any of embodiments 14-16 is disclosed, wherein the train-test splits are generated using the number of different subjects and/or the number of points in time at which brain digital images of the same subject are collected.
18. In an embodiment, the method of any of embodiments 14-16 is disclosed, wherein the train-test splits are generated using the number of different subjects and the number of points in time at which brain digital images of the same subject are collected.
19. In an embodiment, the method of any of embodiments 14-16 is disclosed, wherein the train-test splits are generated using the number of different subjects or the number of points in time at which brain digital images of the same subject are collected.
20. In an embodiment, the method of any preceding embodiments is disclosed, wherein the train-test splits are randomly generated.
21. In an embodiment, the method of any preceding embodiments is disclosed, wherein the step of calculating, for the at least two generated train-test splits and for the one or more annotated features, the divergence between the probability distributions estimated for the pair of the train set and the test set comprises calculating, for the at least two generated train-test splits and for the one or more annotated features, the similarity between the probability distributions estimated for the pair of the train set and the test set, and wherein the step of aggregating, across the one or more annotated features, the calculated divergence for the at least two generated train-test splits comprises aggregating, across the one or more annotated features, the calculated similarity for the at least two generated train-test splits, and wherein the step of selecting the train-test split with the lowest aggregated divergence comprises selecting the train-test split with the highest aggregated similarity.
22. In an embodiment, the method of any preceding embodiments is disclosed, wherein the step of calculating, for the at least two generated train-test splits and for the one or more annotated features, the divergence between the probability distributions estimated for the pair of the train set and the test set comprises calculating, for the at least two generated train-test splits and for the one or more annotated features, the similarity between the probability distributions estimated for the pair of the train set and the test set.
23. In an embodiment, the method of any preceding embodiments is disclosed, wherein the step of aggregating, across the one or more annotated features, the calculated divergence for the at least two generated train-test splits comprises aggregating, across the one or more annotated features, the calculated similarity for the at least two generated train-test splits.
24. In an embodiment, the method of any preceding embodiments is disclosed, wherein the step of selecting the train-test split with the lowest aggregated divergence comprises selecting the train-test split with the highest aggregated similarity.
25. In an embodiment, a computer-implemented method of training one or more machine-learning models for brain lesion detection is disclosed, the method comprising the steps of:
   a. receiving an input training dataset generated according to any preceding embodiments;
   b. training one or more machine-learning models using the received input training dataset;
   c. outputting the trained one or more machine-learning models.
26. In an embodiment, the method of embodiment 25 is disclosed, further comprising assessing the performance of the outputted trained one or more machine-learning models, wherein assessing the performance comprises evaluating true positives, false positives, false negatives, and combinations and/or aggregations thereof.
27. In an embodiment, the method of embodiment 25 is disclosed, further comprising assessing the performance of the outputted trained one or more machine-learning models.
28. In an embodiment, the method of any of embodiments 25-27 is disclosed, further comprising selecting at least one of the one or more trained machine-learning models using the assessed performance.
29. In an embodiment, a method of using one or more machine-learning models, trained according to any of the embodiments 25-28, is disclosed, the method comprising the steps of:
   a. receiving an input brain digital image;
   b. detecting brain lesions in the received input image;
   c. outputting the detected brain lesions.
30. In an embodiment, the method of embodiment 29 is disclosed, wherein the one or more trained machine-learning models comprise a first model and a second model, and wherein outputting the detected brain lesions comprises obtaining first brain lesions detected by the first model, obtaining second brain lesions detected by the second model, and merging the first brain lesions and second brain lesions, in particular wherein the first model is an nnDetection model and the second model is a nnUNet model.
31. In an embodiment, the method of embodiment 29 is disclosed, wherein the one or more trained machine-learning models comprise a first model and a second model.
32. In an embodiment, the method of embodiment 29 is disclosed, wherein the one or more trained machine-learning models comprise a first model and a second model, in particular wherein the first model is an nnDetection model and the second model is a nnUNet model.
33. In an embodiment, a method of monitoring and/or predicting brain lesions in a subject is disclosed, the method comprising using the computer-implemented method of any preceding embodiments, in particular wherein the subject is undergoing or has undergone a treatment.
34. In an embodiment, a method of monitoring and/or predicting brain lesions in a subject is disclosed, the method comprising using the computer-implemented method of any preceding embodiments.
35. In an embodiment, a method of monitoring and predicting brain lesions in a subject is disclosed, the method comprising using the computer-implemented method of any preceding embodiments.
36. In an embodiment, a method of monitoring or predicting brain lesions in a subject is disclosed, the method comprising using the computer-implemented method of any preceding embodiments.
37. In an embodiment, a method of monitoring and predicting brain lesions in a subject is disclosed, the method comprising using the computer-implemented method of any preceding embodiments, in particular wherein the subject is undergoing or has undergone a treatment.
38. In an embodiment, a method of monitoring or predicting brain lesions in a subject is disclosed, the method comprising using the computer-implemented method of any preceding embodiments, in particular wherein the subject is undergoing or has undergone a treatment.
39. In an embodiment, a computer program [product] is disclosed, comprising instructions which, when the program is executed by a computer, cause the computer to carry out the steps of the method of any preceding embodiments.
40. In an embodiment, a system is disclosed, comprising:
   a. a processor; and
   b. a computer readable medium comprising instructions that, when executed by the processor, cause the processor to perform the steps of the method of any preceding embodiments;
   c. optionally a brain digital images acquisition means.
41. In an embodiment, a system is disclosed, comprising:
   a. a processor; and
   b. a computer readable medium comprising instructions that, when executed by the processor, cause the processor to perform the steps of the method of any preceding embodiments.
42. The invention as hereinbefore described.

The present disclosure includes the combination of the aspects and preferred features as described except where such a combination is clearly impermissible or expressly avoided. Where lists are part of the aspects and preferred features as described, the present disclosure includes the combination of the elements of such lists as well as the individual elements of the list as alternatives.

It must be noted, as used in the specification and the appended claims, the singular forms ‘a’, ‘an’, and ‘the’ include plural referents unless the context clearly dictates otherwise. Throughout this specification, including the claims which follow, unless the context requires otherwise, the word ‘comprise’ and variations such as ‘comprises’ and ‘comprising’, will be understood to imply the inclusion of a stated integer or step or group of integers or steps but not the exclusion of any other integer or step or group of integers or steps.

The section headings used herein are for organizational purposes only and are not to be construed as limiting the subject matter described.

### REFERENCES

¹Yoo, S. K. et al (2022), Deep-Learning-Based automatic detection and segmentation of brain metastases with small volume for stereotactic ablative radiotherapy, Cancers (Basel), 14(10):2555.
²Park, Y. W. et al (2021), Radiomics and Deep Learning in brain metastases: current trends and roadmap to future applications, Investig. Magn. Reson. Imaging, 25(4):266-280.
³Juenger, S. T. et al (2021), Fully automated MR detection and segmentation of brain metastases in non-small cell lung cancer using Deep Learning, J. Magn. Reson. Imaging, 54(5):1608-1622.
⁴Bousabarah, K. et al (2020), Deep convolutional neural networks for automated segmentation of brain metastases trained on clinical data, Radiation Oncology 15:87.
⁵Xue, J. et al (2020), Deep-learning-based detection and segmentation-assisted management of brain metastases, Neuro Oncol. 22(4):505-514.
⁶Zhou, Z. et al (2020), Computer-aided detection of brain metastases in T1-weighted MRI for stereotactic radiosurgery using Deep Learning single-shot detectors, Radiology, 295 (2): 407-415.
⁷Charron, O. et al (2018), Automatic detection and segmentation of brain metastases on multimodal MR images with a deep convolutional neural network, Comput. Biol. Med., 95:43-54.
⁸Grovik, E. et al (2020), Deep learning enables automatic detection and segmentation of brain metastases on multisequence MRI, J. Magn. Reson. Imaging, 51 (1): 175-182.
⁹Liu, Y. et al (2017), A deep convolutional neural network-based automatic delineation strategy for multiple brain metastases stereotactic radiosurgery, PLOS One, 12 (10): e0185844.
¹⁰Huang, Y. et al (2022), Deep learning for brain metastasis detection and segmentation in longitudinal MRI data, Med. Phys. 49 (9): 5773-5786.
¹¹https://pubmed.ncbi.nlm.nih.gov/28586279/.

TABLE I
statistics of selected patients.

| | Number of | | | Prior radiation |
|---|---|---|---|---|
| Sex | patients | Average age | Non-smokers | treatment |
| Female | 49 | 52.9 | 36 | 21 |
| Male | 38 | 57.2 | 18 | 18 |

TABLE II
statistics of slice thickness values for different MR sequences.

| Slice thickness | | | | |
|---|---|---|---|---|
| [mm] | T1CE | T1 | T2 | FLAIR |
| Min | 0.5 | 0.5 | 1 | 0.5 |
| Mean | 1 | 4.46 | 5.44 | 5.31 |
| Std | 0.2 | 2.12 | 1.28 | 1.25 |
| Max | 1.5 | 8.25 | 13 | 8.25 |

TABLE III
statistics of total volumes of the annotations.

| | Enhancing | | | |
|---|---|---|---|---|
| [mm³] | tumor | Necrosis | Edema | Cavity |
| Mean | 2144.0 | 1406.1 | 9491.1 | 987.7 |
| Min | 0.0 | 0.0 | 0.0 | 0.0 |
| Quantile 0.25 | 38.9 | 0.0 | 0.0 | 0.0 |
| Quantile 0.50 | 417.7 | 75.0 | 1001.3 | 0.0 |
| Quantile 0.75 | 1631.4 | 1103.0 | 7175.6 | 0.0 |
| Max | 44543.0 | 28712.3 | 154210.0 | 86463.0 |

TABLE IV
statistics of the number of visits across patients.

| Visits count | Number of patients |
|---|---|
| 1 | 19 |
| 2 | 12 |
| 3 | 31 |
| 4 | 13 |
| 5 | 3 |
| 6 | 3 |
| 8 | 3 |
| 9 | 2 |
| 12 | 1 |

TABLE V
statistics of the stratification features.

| | Nec_to_et + | Et_nec_blob_{—} | Et_nec_mean_{—} | Cav_tot_{—} | Ed_tot_{—} |
|---|---|---|---|---|---|
| [mm³] | nec | count | volume | volume | volume |
| Mean | 32.68 | 11.67 | 761.30 | 978.72 | 9491.12 |
| Min | 0.00 | 0.00 | 3.65 | 0.00 | 0.00 |
| Quantile | 0.00 | 1.00 | 25.87 | 0.00 | 0.00 |
| 0.10 | | | | | |
| Quantile | 0.00 | 1.00 | 70.75 | 0.00 | 0.00 |
| 0.25 | | | | | |
| Quantile | 11.39 | 3.00 | 242.81 | 0.00 | 1001.25 |
| 0.50 | | | | | |
| Quantile | 67.78 | 9.00 | 754.85 | 0.00 | 7175.58 |
| 0.75 | | | | | |
| Quantile | 100.00 | 25.60 | 1940.56 | 0.00 | 27981.86 |
| 0.90 | | | | | |
| Max | 100.00 | 312.00 | 17155.00 | 86463.00 | 154210.00 |

TABLE VI
statistics of the number of visits for patients
in the train set and in the test set.

| Visits counts | Test set patients | Train set patients |
|---|---|---|
| 1 | 6 | 13 |
| 2 | 0 | 12 |
| 3 | 6 | 25 |
| 4 | 3 | 10 |
| 5 | 1 | 2 |
| 6 | 2 | 1 |
| 8 | 0 | 3 |
| 9 | 0 | 2 |
| 12 | 1 | 0 |

1. A computer-implemented method of generating a training dataset for a machine-learning model for brain lesion detection, comprising the steps of:
a. receiving (20) an input dataset of brain digital images, comprising annotated features;
b. generating (22) at least two train-test splits of the received input dataset, wherein each train-test split consists of a split of the dataset in a pair of a train set and a test set;
c. estimating (24), for the at least two generated train-test splits, one or more probability distributions associated with one or more of the annotated features in the train set and one or more probability distributions associated with one or more of the annotated features in the test set;
d. calculating (26), for the at least two generated train-test splits and for the one or more annotated features, the divergence between the probability distributions estimated for the pair of the train set and the test set;
e. aggregating (28), across the one or more annotated features, the calculated divergence for the at least two generated train-test splits;
f. selecting (30) the train-test split with the lowest aggregated divergence;
g. outputting (32) the train set of the selected train-test split.

2. The method of claim 1, wherein the annotated features comprise: number of enhancing tumor lesions, number of necrosis, number of edema regions, number of cavities, volume of enhancing tumor lesions, volume of necrosis, volume of edema regions, volume of cavities, and any combinations and/or aggregations thereof.

3. The method of claim 1, wherein the input dataset comprises brain digital images from MRI scans collected with sequences comprising: TICE, T1, T1-FLAIR, T2, T2-FLAIR.

4. The method of claim 1, wherein the input dataset comprises brain digital images of different subjects and/or brain digital images of the same subject collected at different points in time.

5. The method of claim 4, wherein the train-test splits are generated using the number of different subjects and/or the number of points in time at which brain digital images of the same subject are collected.

6. The method of claim 1, wherein the train-test splits are randomly generated.

7. The method of claim 1, wherein the step of calculating, for the at least two generated train-test splits and for the one or more annotated features, the divergence between the probability distributions estimated for the pair of the train set and the test set comprises calculating, for the at least two generated train-test splits and for the one or more annotated features, the similarity between the probability distributions estimated for the pair of the train set and the test set, and wherein the step of aggregating, across the one or more annotated features, the calculated divergence for the at least two generated train-test splits comprises aggregating, across the one or more annotated features, the calculated similarity for the at least two generated train-test splits, and wherein the step of selecting the train-test split with the lowest aggregated divergence comprises selecting the train-test split with the highest aggregated similarity.

8. A computer-implemented method of training one or more machine-learning models for brain lesion detection, comprising the steps of:
a. receiving an input training dataset generated according to claim 1;
b. training one or more machine-learning models using the received input training dataset;
c. outputting the trained one or more machine-learning models.

9. The method of claim 8, further comprising assessing the performance of the outputted trained one or more machine-learning models, wherein assessing the performance comprises evaluating true positives, false positives, false negatives, and combinations and/or aggregations thereof.

10. The method of claim 9, further comprising selecting at least one of the one of more trained machine-learning models using the assessed performance.

11. A method of using one or more machine-learning models, trained according to claim 8, to detect brain lesions, the method comprising the steps of:
a. receiving an input brain digital image;
b. detecting brain lesions in the received input image;
c. outputting the detected brain lesions.

12. The method of claim 11, wherein the one or more trained machine-learning models comprise a first model and a second model, and wherein outputting the detected brain lesions comprises obtaining first brain lesions detected by the first model, obtaining second brain lesions detected by the second model, and merging the first brain lesions and second brain lesions, in particular wherein the first model is an nnDetection model and the second model is a nnUNet model.

13. A method of monitoring and/or predicting brain lesions in a subject, the method comprising using the computer-implemented method of claim 1, in particular wherein the subject is undergoing or has undergone a treatment.

14. A computer program comprising instructions which, when the program is executed by a computer, cause the computer to carry out the steps of the method of claim 1.

15. A system comprising:
a. a processor; and
b. a computer readable medium comprising instructions that, when executed by the processor, cause the processor to perform the steps of the method of claim 1;
c. optionally a brain digital images acquisition means.
